(19) United States
(12) Patent Application Publication
(10) Pub. No.: US 2007/0148154 A1
(43) Pub. Date: Jun. 28, 2007

(54) Use of superoxide dismutase mimetics and reductase gultatione in the form of anticancer drugs

(75) Inventors: Bernard Weill, Esubonne (FR); Frederic Batteux, Paris (FR); Alexis Laurent, Saint Maurice (FR)

Correspondence Address: OBLON, SPIVAK, MCCLELLAND, MAIER & NEUSTADT, P.C., 1940 DUKE STREET, ALEXANDRIA, VA, 22314, US

(73) Assignees: UNIVERSITE RENE DESCARTES (PARIS V), PARIS (FR); PROTEXEL, PARIS (FR)

(21) Appl. No.: 10/583,135

(22) PCT Filed: Dec. 17, 2004
(86) PCT No.: PCT/FR04/03298

§ 371 Date: Dec. 26, 2006

(51) Int. Cl. A61K 38/44; A61K 38/14; A61K 31/7072; A61K 31/704; A61K 31/555; A61K 31/4745; A61K 31/337; A61K 31/282

(52) U.S. Cl. 424/94.4; 424/649; 514/34; 514/185; 514/492; 514/49; 514/27; 514/283; 514/410; 514/449; 514/8

(57) ABSTRACT

The invention relates to the use of superoxide dismutase mimetics and reductase glutathione (preferably Mangafodipir) in order to produce an anticancer drug. Said mimetic, in particular makes it possible to potentiate the actions of antitumoral agents generating active oxygen forms on tumoral cells and to protect non-tumoral cells against said actions.

The present invention relates to the use of chemical mimetics of superoxide dismutase (SOD) for inhibiting tumor growth, and potentiating the effects of antitumor treatments on tumor cells while at the same time inhibiting the toxic effects thereof on normal cells.

The term “reactive oxygen species” (ROS) encompasses a set of reduced oxygen derivatives, such as the superoxide anion (O₂⁻), hydrogen peroxide (H₂O₂) or the hydroxyl radical (OH⁺). These derivatives are normally generated by cellular metabolism, in particular in the mitochondria, during the reduction of molecular oxygen to H₂O. They are also produced in large amounts under certain conditions, for example during exposure to ionizing radiation or to ultraviolet rays, or during exposure to certain chemical products.

Since reactive oxygen species are very toxic, cells of various means of neutralizing them. Among these detoxification means are in particular “antioxidant” enzymes, among which mention will be made of superoxide dismutases (SOD; EC 1.15.1.1) which catalyze the dismutation of the superoxide anion to hydrogen peroxide+O₂, and the enzymes subsequently involved in the detoxification of the hydrogen peroxide, such as the catalyze (EC 1.11.1.6) which catalyzes the dismutation of hydrogen peroxide (2H₂O₂→O₂+2H₂O), glutathione peroxidase (EC 1.11.1.9) which catalyzes the reduction of hydrogen peroxide by reduced glutathione (GSH), producing oxidized glutathione (GSSG) and water (2 GSH+H₂O₂→GSSG+2H₂O), and glutathione reductase (EC 1.8.1.7), which regenerates GSH according to the reaction GSSG+NADPH+H⁺→2 GSH+NADP⁺.

When the production of reactive oxygen species exceeds the cell's detoxification capacities, the toxic effects of these derivatives manifest themselves, and can induce considerable damage to cell constituents such as proteins, membrane lipids or DNA. The oxidative stress thus generated plays a major role in the appearance and the development of various diseases, in particular inflammatory and autoimmune pathologies, and cancers.

It is at the current time generally accepted that reactive oxygen species are involved in the pathogenesis of many cancers. However, it appears that their effects involve complex mechanisms which are far from being elucidated.

In sublethal amounts, ROSs can promote the appearance of cancers, for example by causing mutations in coding regions or regulatory regions, or by inhibiting or, conversely, stimulating the expression of genes involved in the regulation of cell proliferation or differentiation, or of apoptosis. It has thus been proposed to use antioxidants in the context of curative or preventive treatments for various cancers. For example, a diet supplemented with antioxidants, in particular with vitamin E, has been recommended with the aim of preventing cancer.

At high concentrations, ROSs can directly induce cell death, in particular by causing lipid and protein peroxidation reactions, which can promote mitochondrial depolarization and thus accelerate the effector phases of apoptosis. This activation of apoptosis by ROSs can constitute a means of destroying tumor cells.

For example, radiotherapy treatments are based essentially on the induction of an overproduction of ROSs in tumor cells. Similarly, many molecules used in cancer chemotherapy induce an overproduction of ROSs in the cells, which would be responsible, at least in part, for the antitumor effect of these molecules.

Anticancer molecules that can induce ROS production can belong to various therapeutic classes. Mention will in particular be made of intercollating agents, for example anthracyclines such as doxorubicin which inhibits replication and induces DNA damage; topoisomerase-2 inhibitors such as etoposide which induces DNA breakages; antimetabolites such as 5-fluorouracil; electrophilic agents such as mitomycin C and platinum derivatives [cisplatin (YOKOMIZO et al., Cancer Res, 55: 4293-4296 1995) and oxaliplatin]; spindle poisons such as taxanes; and anti-hormone receptors such as tamoxifen (FERLINI et al., Br J Cancer, 79, 257-263, 1999).

However, one of the main limitations to the use of these anticancer molecules is due to the fact that their action can also lead to the death of normal cells and cause lesions, sometimes irreversible, with very prejudicial consequences.

Most anticancer molecules preferentially destroy rapidly dividing cells. Their toxicity with respect to normal cells is therefore generally less than with respect to tumor cells. However, there exist, in certain tissues, cells whose division rate is very rapid, and which are therefore particularly sensitive to the toxic effects of anticancer agents. These are in particular differentiating haematopoietic cells of the bone marrow. Myelotoxicity constitutes the most common of the toxicities associated with chemotherapy and is associated with the majority of antitumor treatments. It affects essentially leukocytes and platelets, and is reflected in particular by leucopenia, which increases the risk of infection in treated patients.

Certain anticancer molecules also exhibit cytotoxicity that targets more specifically certain tissues or organs. By way of examples: anthracyclines, such as doxorubicin, have a cardiotoxic effect which would result in the production of ROSs, leading to peroxidation of the lipid structures of the sarcoplasmic reticulum and of the mitochondria, and a dysfunction of these organelles; bleomycin has a strong pulmonary toxicity, also attributed to the production of ROSs, and which can result in irreversible interstitial pulmonary fibrosis.

Various strategies for decreasing the side effects of anticancer treatments have been proposed.

In the case of a cytotoxicity concerning more particularly certain cell types, it has been proposed to use cytoprotective agents, and in particular agents capable of neutralizing ROSs, such as N-acetylcysteine (DOROSHOW et al., J. Clin. Invest., 68, 1053-1064, 1981) or, more recently, SOD or mimetics of this enzyme. For example, application PCT/WO 97/49390 proposes the use of a manganese chelate derived from dipyrydoxal, MnDPDP, for preventing the cardiotoxic effects of anthracyclines; application PCT/WO 02/060383 reports the ability of two manganese chelates derived from porphyrin, MnTBAP and MnTM-4-PyP, to protect the cells of the pulmonary epithelium against the toxic effects of radiotherapy and of bleomycin; this application also reports that these derivatives are capable of selectively inhibiting the proliferation of pulmonary adenocarcinoma cells, without affecting that of normal epithelial or endothelial cells.

In order to reduce the consequences of the cytotoxic effects of anticancer molecules with respect to haematopoietic cells, haematopoietic growth factors are generally used in order to reduce the period of leucopenia and the risk of infection which ensues therefrom. The use of cytoprotective agents is limited by the risk of the lack of selectivity of these agents, due to the rapid division rate of haematopoietic cells. At the current time, the only cytoprotective agent used to reduce the leucopenia is amiphostine, which is a phosphorylated precursor of an antioxidant containing a thiol group, the selectivity of which results from its preferential penetration into nontumor cells, where it releases the active molecule.

The inventors undertook to test the effects of various molecules, known for their ability to neutralize, at various levels, ROS production, on the proliferation of various tumor cell lines and also on the viability of these tumor cells and that of normal human leukocytes; they subsequently tested, in the same manner, the effects of these molecules on the cytostatic and cytotoxic properties of antitumor chemotherapy agents known to induce ROS production.

The antioxidant molecules which were tested are as follows:
N-acetylcystein (NAC), which is an antioxidant that is a free-radical scavenger and precursor of intracellular glutathione;
CuDIPS (Cu[II]-[diisopropylsalicylate]), which is a chemical mimetic of CuZn SOD (MC KENZIE et al., Br. J. Pharmacol. 127, 1159-1164, 1999);
MnTBAP (Mn(III) tetrakis(5,10,15,20-benzoic acid)-porphyrin), which is a chemical mimetic of MnSOD (PASTERNACK et al., Inorg. Biochem., 15, 261-267 1981) and also catalase and glutathione peroxidase (application PCT/WO 01/12327);
MnDPDP (manganese dipyridoxyl phosphate (Mn-DPDP), also called mangafodipir (INN)), which is a chemical mimetic of MnSOD and also of catalase and of glutathione reductase (application PCT/WO 02/087579).

The inventors have observed that treatment with NAC induces an increase in tumor cell proliferation, whereas treatment with MnTBAP, CuDIPS or MnDPDP induces a reduction in this proliferation. As regards cell viability, NAC has no effect thereon, whether tumor cells or normal human leukocytes are involved. MnTBAP or CuDIPS decreases tumor cell viability and also, although to a lesser extent, that of normal human leukocytes. On the other hand, MnDPDP decreases tumor cell viability, but, surprisingly, does not influence that of normal human leukocytes.

In the case of the combination of these antioxidant molecules with antitumor agents, the inventors have observed that NAC inhibits the cytostatic and cytotoxic effects of these agents on tumor cells, whereas MnTBAP, CuDIPS and MnDPDP increase them.

The effects of NAC, of MnTBAP and of CuDIPS on the cytotoxicity of antitumor agents with respect to normal leukocytes are similar to those observed on tumor cells; on the other hand, MnDPDP decreases the cytotoxicity of antitumor agents on normal human leukocytes, conversely to the effect observed in the case of tumor cells.

It therefore appears that MnDPDP is capable of inducing or potentiating a chemo-induced oxidative stress in tumor cells, while at the same time preserving the viability of normal leukocytes.

The inventors have also tested the effects of NAC, of MnTBAP, of CuDIPS and of MnDPDP, administered on their own or combined with an antitumor chemotherapy agent, on the development of tumors in vivo in mice.

They have observed that the administration of NAC induces an increase in tumor volume, whereas the administration of MnTBAP, of CuDIPS or of MnDPDP decreases the tumor volume. In combination with an antitumor agent, NAC blocks the inhibitory effect of this agent on tumor growth, whereas MnTBAP, CuDIPS or MnDPDP increases this inhibitory effect.

These singular properties of mangafodipir, compared with those of other antioxidants, and in particular of the other SOD mimetics tested, appear to be linked to its double activity of superoxide dismutase mimetic and glutathione reductase mimetic.

A subject of the present invention is the use of a superoxide dismutase and glutathione reductase mimetic as an antitumor and leukocyte-protecting active ingredient, for obtaining an anticancer medicinal product.

SOD mimetics also having a glutathione reductase mimetic activity, that can be used in accordance with the invention, are in particular dipyridoxal phosphate derivatives such as those described in patent EP 0936615, in the form of the divalent cation chelates thereof, such as copper chelates, zinc chelates or, advantageously, manganese chelates.

More generally, any molecule which has an SOD mimetic activity, and which is also capable of mimicking glutathione reductase by reducing oxidized glutathione, can be used.

According to a preferred embodiment of the present invention, said superoxide dismutase and glutathione reductase mimetic is mangafodipir (MnDPDP).

According to a preferred embodiment of the present invention, said superoxide dismutase and glutathione reductase mimetic is used in combination with another antitumor agent, preferably an antitumor agent capable of inducing an ROS production in cells.

By way of examples of antitumor agents capable of inducing, in cells, an ROS production, that can be used in the context of the present invention, mention will in particular be made, in addition to the antitumor agents mentioned above (doxorubicin, mitomycin C, etoposide, platinum derivatives, tamoxifen, taxanes, 5-fluorouracil), of the following molecules: irinotecan (topoisomerase-1 inhibitor), gemcitabine (antimetabo-lite), endoxan (electrophilic alkylating agent), streptozotocin (electrophilic nonalkylating agent), bleomycin (DNA-cleaving agent) and vincristine (spindle poison).

Because of the simultaneous nature of their cytotoxic and cytostatic effect with respect to tumor cells, and their protective effect with respect to normal leukocytes, the superoxide dismutase and glutathione reductase mimetics make it possible to significantly increase the therapeutic index of the anticancer medicinal products with which they are combined. In fact, they exert, with these anticancer medicinal products, a synergistic antitumor action, while at the same time protecting the leukocytes against the harmful effects of the chemotherapy.

A subject of the present invention is also a pharmaceutical composition comprising mangafodipir combined with another antitumor agent, as defined above.

For the implementation of the present invention, the mangafodipir will generally be used in formulations for the administration of a dose of active ingredient of between 1 and 100 mg/kg/day. Higher doses can, however, be used, given the low toxicity of this product. It is clearly understood that those skilled in the art can adjust these doses according to the particularities of each patient and the pathology concerned.

These formulations can be administered by various routes, for example orally, or by means of injections, in particular subcutaneous, intramuscular or intravenous injections. Other routes of administration may be envisioned if they increase the effectiveness, the bioavailability or the tolerance of the products. The most appropriate route can be chosen by those skilled in the art according to the formulation used.

The present invention will be understood more clearly from the further description which follows, which refers to nonlimiting examples showing the antitumor properties of mangafodipir and its cytoprotective effects on normal leukocytes.

### EXAMPLE 1

### Influence of Various Antioxidant Molecules On the Basal Proliferative Properties of Tumor Cells

In vitro cell proliferation assays were carried out on the following cell lines: CT26 (mouse colon carcinoma, ATCC (American Type Culture Collection) No. 2638), Hepa 1-6 (mouse liver hepatoma, ATCC No. 1830), A549 (human lung carcinoma, ATCC No. 185). These lines were cultured beforehand in a humid incubator at 37° C. under 5% of CO₂, in Dulbecco's modified Eagle's medium (DMEM/Glutamax-I containing 10% of fetal calf serum and antibiotics [penicillin (100 U/ml)/streptomycin (100 μg/ml)] (LIFE TECHNOLOGIES, Cergy Pontoise, France). All these cell lines were tested regularly in order to exclude any mycoplasmic infections.

For the proliferation assay, the cells (2×10⁴ cells/well) were seeded into 96-well plates (COSTAR, Corning Inc. NY, USA) and incubated for 48 hours in complete medium supplemented with increasing concentrations, from 0 to 400 μm, of N-acetylcysteine (NAC, SIGMA, Saint-Quentin Fallavier, France), of MnTBAP (MnSOD mimetic; CALBIOCHEM, Paris, France), of CuDIPS (Cu/Zn SOD mimetic; SIGMA, Saint-Quentin Fallavier, France) or of mangafodipir (MnDPDP or TESLASCAN, AMERSHAM HEALTH, Amersham, UK).

The cell proliferation is determined by incubating the cells for 16 hours with [³H]-thymidine (1 μCi/well).

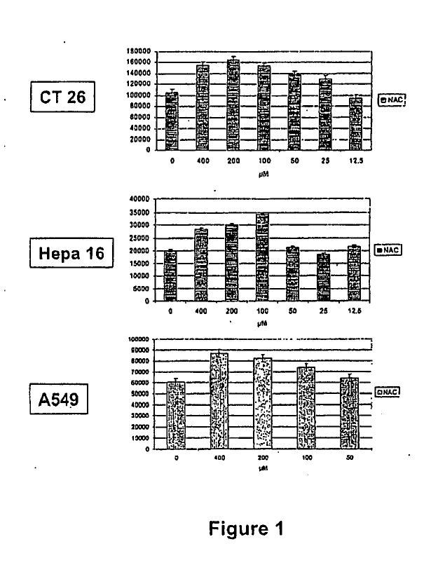

The results of these experiments, on various tumor lines, for NAC, MnTBAP, CuDIPS and MnDPDP, are given in FIGS. 1, 2, 3 and 4, respectively.

Legend of FIGS. 1, 2, 3 and 4:

along the x-axis: concentration of antioxidant (in μM),

along the y-axis: [³H]-thymidine radioactivity in cpm.

An increase in the tumor cell proliferation is observed in response to the treatment with NAC (FIG. 1). This increase in proliferation is 73% for the Hepa 1-6 cells, in the presence of 100 μM of NAC, and 45% and 47% in the presence of 400 μM of NAC for the A549 and CT26 tumor cells, respectively.

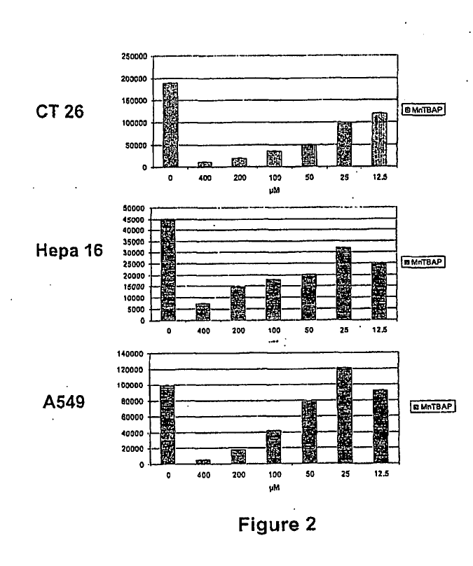

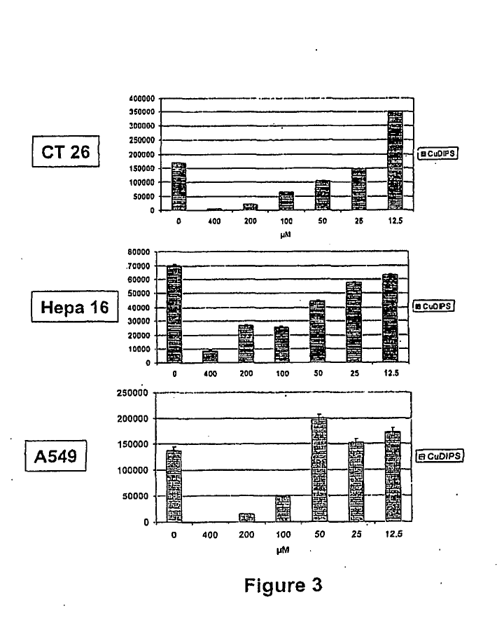

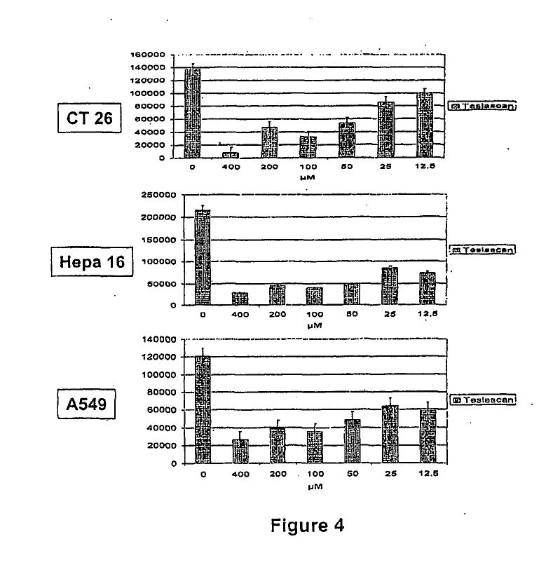

Conversely, the treatment of the Hepa 1-6, CT26 and A549 tumor cells with MnTBAP (FIG. 2), CuDIPS (FIG. 3) or MnDPDP (TESLASCAN, FIG. 4) reduces the proliferation thereof in a dose-dependent manner. This reduction in cell proliferation reaches close to 90% in the presence of 400 μM of one of these three molecules.

### EXAMPLE 2

### Effects of NAC, of CuDIPS, of MnTBAP and of MnDPDP on the Viability of Tumor Lines or of Normal Human Leukocytes

In vitro viability tests, in response to the treatment with NAC, CuDIPS, MnTBAP or MnDPDP, were carried out on the cell lines of Example 1 and on normal human leukocytes. The latter were obtained from normal volunteers, after informed consent, by taking venous blood samples collected on an anticoagulant (lithium-heparinate). The red blood cells were lysed by osmotic shock using a hypotonic solution of potassium acetate and the leukocytes were cultured under the conditions described in Example 1.

For the viability test, the cells (2×10⁴ cells/well) were seeded into 96-well plates (COSTAR, Corning Inc. NY, USA) and incubated for 48 hours in complete medium supplemented with increasing concentrations, from 0 to 400 μm, of NAC, of MnTBAP, of CuDIPS or of MnDPDP. The cell viability was evaluated by reduction of a methylthiazoletetrazolium salt (MTT; SIGMA) into formazan. The cells were exposed to 20 μl of MTT (5 mg/ml in PBS) and incubated for 4 h at 37° C. 150 μl of medium were then removed from each well and the reaction was visualized by the addition of 100 μl of DMSO (SIGMA). The absorbance was analyzed for each well at 550 nm and at 630 nm with an ELISA plate reader. The number of viable cells was determined by the difference between the absorbance at 550 nm and the absorbance at 630 nm.

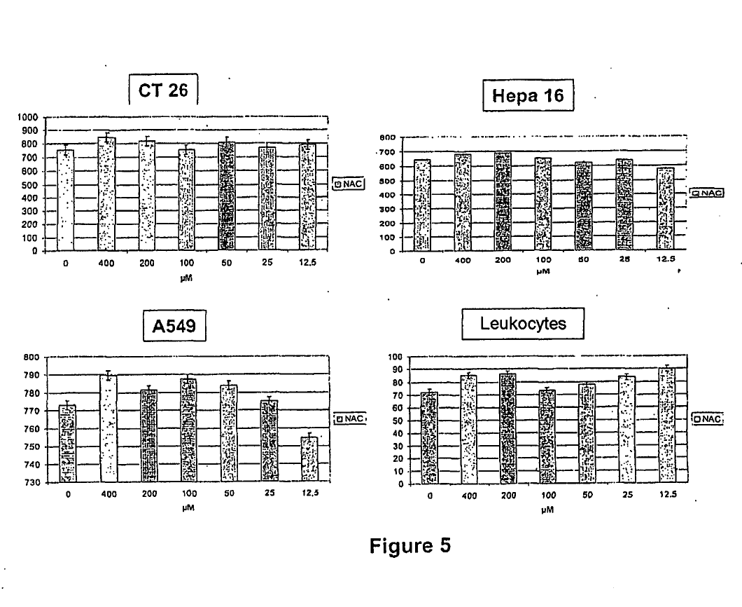

The results of these experiments for the CT26, Hepa 16 and A549 tumor lines, and for the normal leukocytes, are given in FIGS. 5, 6, 7 and 8, for NAC, MnTBAP, CuDIPS and MnDPDP, respectively.

Legend of FIGS. 5 to 8:

along the x-axis: concentration of antioxidant (in μM),

along the y-axis: OD at 550 nm—OD at 630 nm.

It is observed that the NAC treatment of the Hepa 1-6, CT26 and A549 tumor cells or of the normal human leukocytes have no effect on the cell viability (FIG. 5).

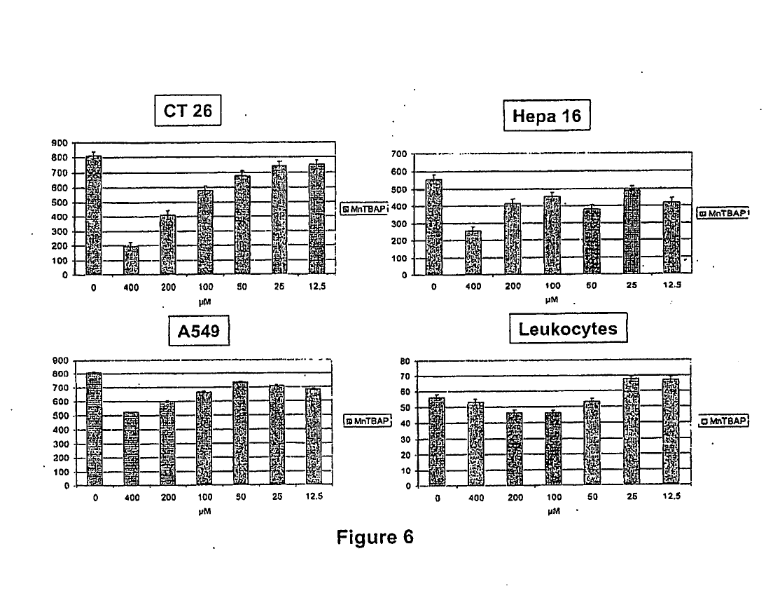

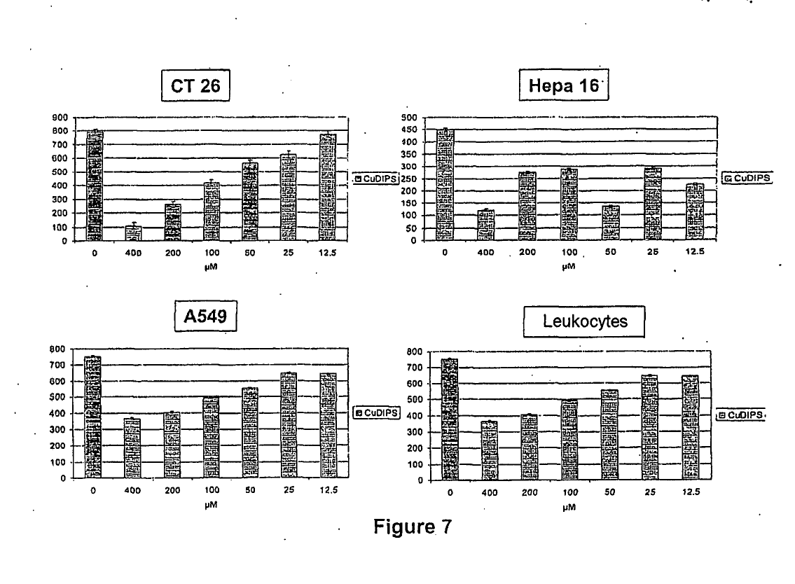

Conversely, the treatment of the Hepa 1-6, CT26 and A549 cells with MnTBAP (FIG. 6) or CuDIPS (FIG. 7) decreases the tumor cell viability in a dose-dependent manner. The viability of the Hepa 1-6, CT26 and A549 tumor cells is reduced by 62%, 75% and 37%, respectively, with 400 μM MnTBAP, and by 74%, 85% and 50%, respectively, with 400 μM of CuDIPS. However, the treatment of normal human leukocytes with MnTBAP and CuDIPS also induces a decrease in cell viability, which reaches a maximum of 18% and 50%, respectively.

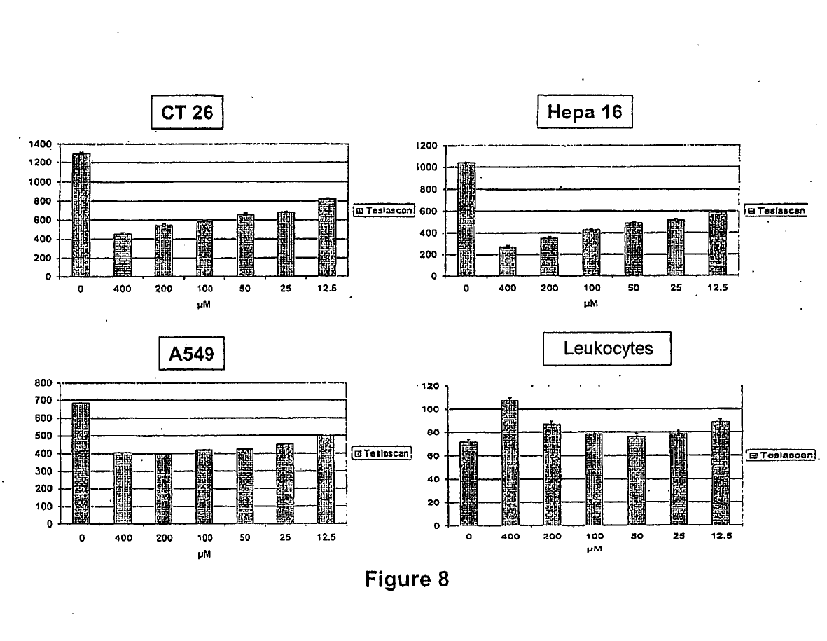

Finally, while MnDPDP (mangafodipir or TESLASCAN, FIG. 8) also reduces, in a dose-dependent manner, the viability of the Hepa 1-6, CT26 and A549 tumor cells, it does not influence the viability of normal human leukocytes, whatever the dose of mangafodipir used.

### EXAMPLE 3

### Effects of NAC, of CuDIPS, of MnTBAP and of MnDPDP on the Antiproliferative and Cytotoxic Properties of Molecules Used in Cancer Chemotherapy

The following antitumor molecules: oxaliplatin (belonging to the cisplatin family); taxol; 5-fluorouracil; which are known to induce ROS production in tumor cells, were used. For each of these molecules, cell proliferation assays and cell viability tests were carried out, in the absence of antioxidant molecules, or in the presence of increasing concentrations of NAC, of MnTBAP, of CuDIPS or of MnDPDP.

1) Effects on the Antiproliferative Properties:

The proliferation assays were carried out on the CT26, Hepa 16 and A549 tumor lines, according to the protocol described in Example 1.

Oxaliplatin:

Oxaliplatin (ELOXATIN or [(1R,2R)-1,2-cyclohexanediamine-N,N′][oxalate-(2-)—O,O′]platinum (II); SANOFI-PHARMA, Paris, France) was used in all the assays at a concentration of 10 μM.

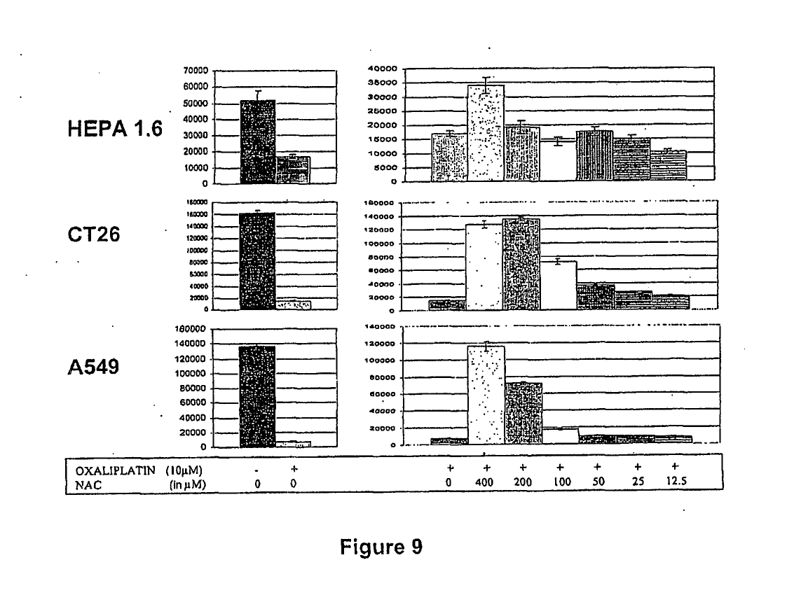

The results of the CT26, Hepa 16 and A549 tumor line cell proliferation assays are given in FIGS. 9, 10, 11 and 12, for NAC, MnTBAP, CuDIPS and MnDPDP, respectively.

Legend of FIGS. 9 to 12:

along the x-axis: presence (+) or absence (−) of

oxaliplatin; concentration of antioxidant (in μM),

along the y-axis: [³H]-thymidine radioactivity in cpm.

The treatment of Hepa 1-6, CT26 and A549 tumor lines with 10 μM of oxaliplatin alone decreases the tumor cell proliferation by 70%, 91% and 93%, respectively (FIGS. 9 to 12).

NAC reduces, in a dose-dependent manner, the cytostatic effect of oxaliplatin, whatever the tumor cell type (FIG. 9).

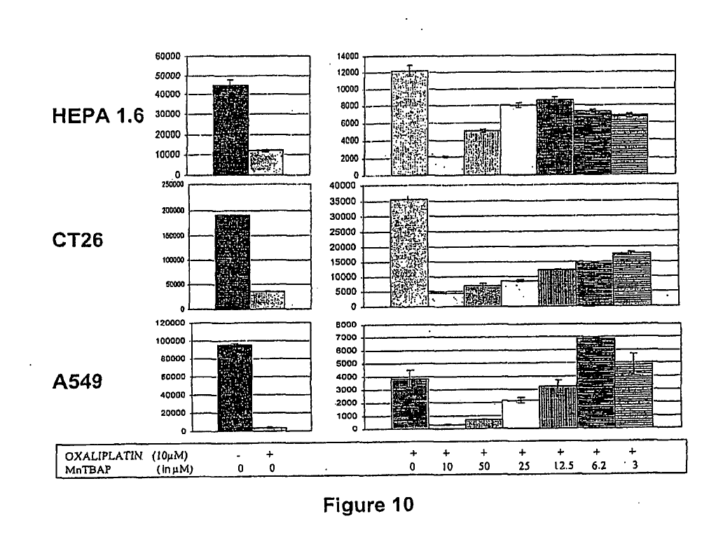

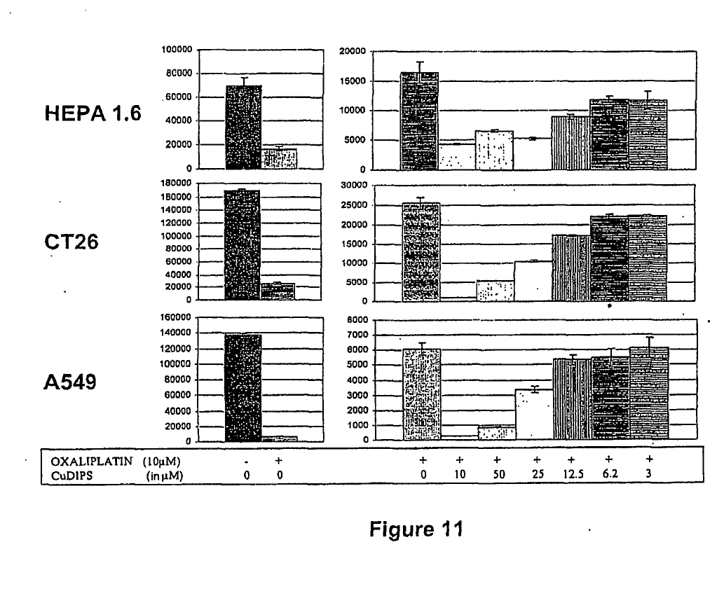

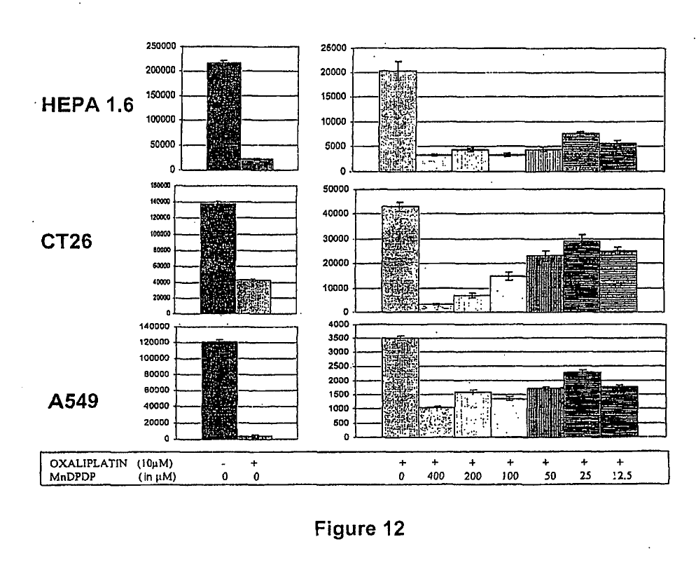

Conversely, MnTBAP (FIG. 10), CuDIPS (FIG. 11) and MnDPDP (FIG. 12) increase, in a dose-dependent manner, the antiproliferative properties of oxaliplatin.

Taxol:

Taxol (PACLITAXEL; BRISTOL-MYERS-SQUIBB, Paris, France) was used in all the assays at a concentration of 10 μM.

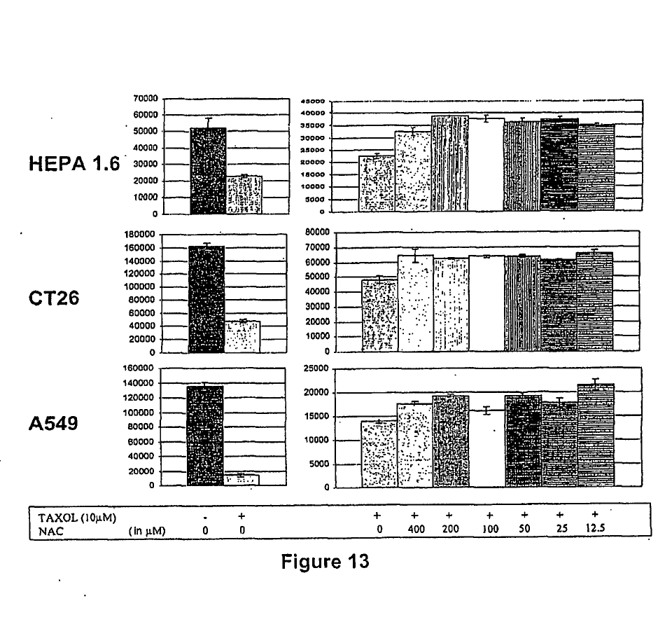

The results of the CT26, Hepa 16 and A549 tumor line proliferation assays are given in FIGS. 13, 14, 15 and 16, for NAC, MnTBAP, CuDIPS and MnDPDP, respectively.

Legend of FIGS. 13 to 16:

along the x-axis: presence (+) or absence (−) of taxol; concentration of antioxidant (in μM),

along the y-axis: [³H]-thymidine radioactivity in cpm.

The incubation with taxol reduces, respectively, the proliferation of the A549, CT26 or Hepa 1-6 tumor cells by 85%, 71% and 65% (FIGS. 13 to 16).

The addition of NAC reduces, in a dose-dependent manner, the cytostatic effect of taxol on the tumor cells (FIG. 13).

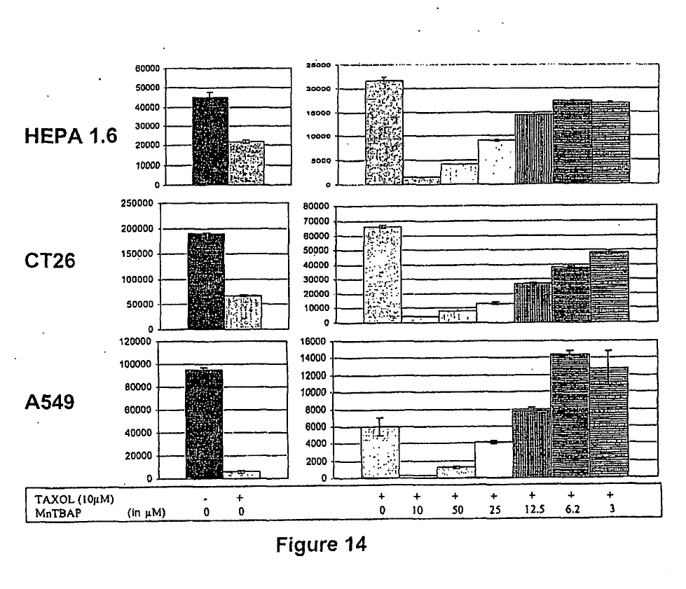

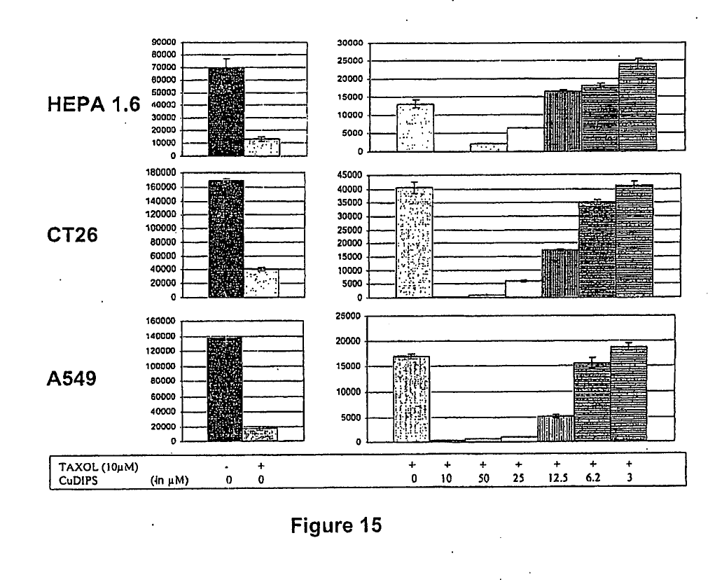

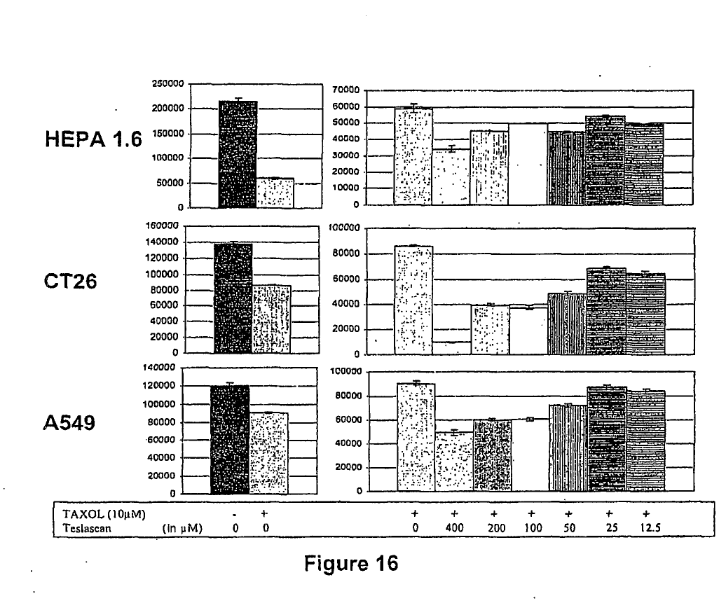

Conversely, the addition of the three SOD mimetics [MnTBAP (FIG. 14), CuDIPS (FIG. 15) or MnDPDP (FIG. 16)] increases the cytostatic effect of taxol in a dose-dependent manner.

5-Fluorouracil (5-FU):

5-Fluorouracil (5-FU) (5-fluoro-1,2,3,4-tetrahydropyrimidine-2,5-dione or fluorouracil; ICN PHARMACEUTICAL FRANCE, Orsay, France) was used in all the assays at a concentration of 50 μM.

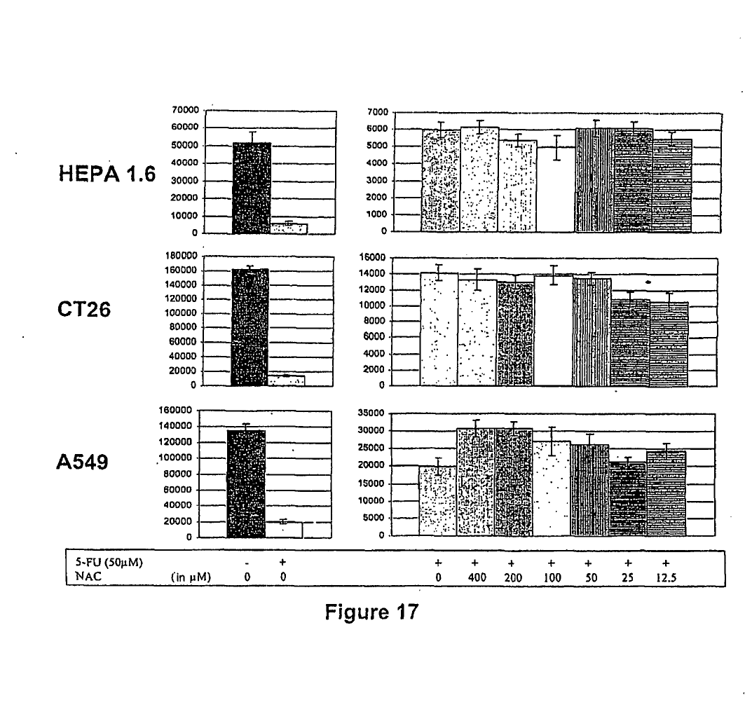

The results of the CT26, Hepa 16 and A549 tumor line proliferation assays are given in FIGS. 17, 18, 19 and 20, for NAC, MnTBAP, CuDIPS and MnDPDP, respectively.

Legend of FIGS. 17 to 20:

along the x-axis: presence (+) or absence (−) of 5-FU; concentration of antioxidant (in μM),

along the y-axis: [³H]-thymidine radioactivity in cpm.

Incubation of the tumor cells with 5-FU reduces the Hepa 1-6, CT26 and A549 tumor cell proliferation by 91%, 91% and 85%, respectively (FIGS. 17 to 20).

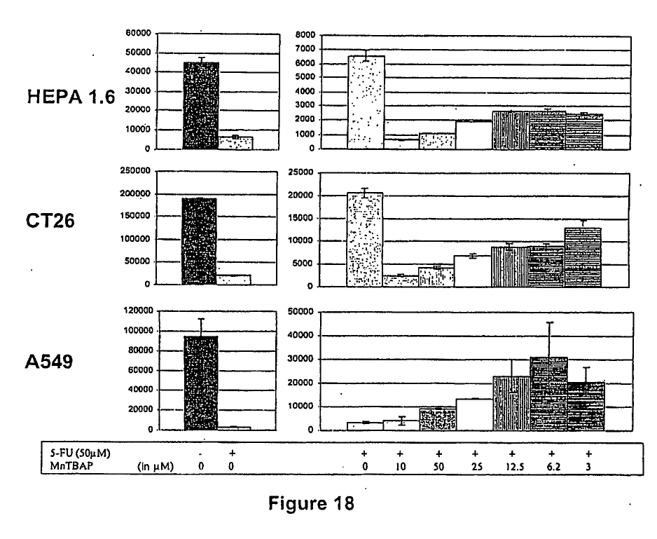

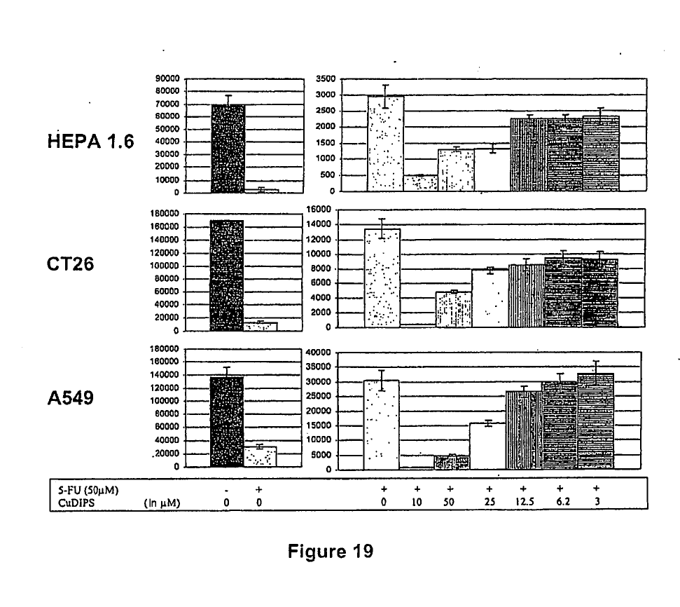

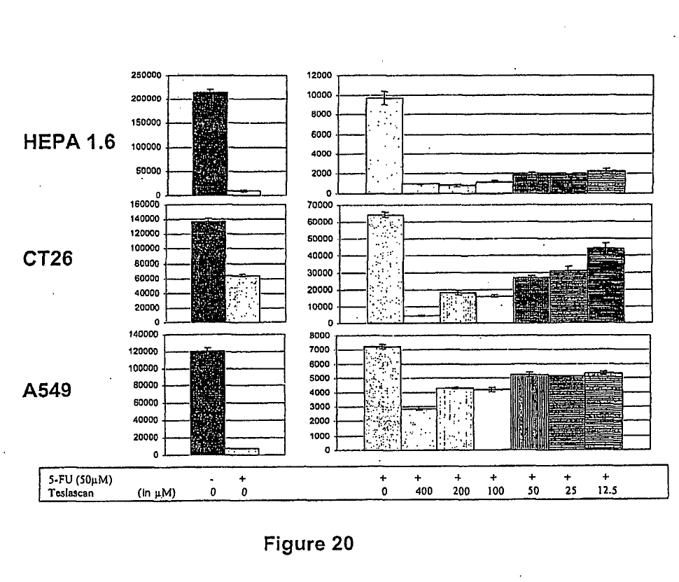

As for oxaliplatin and TAXOL, NAC inhibits the cytostatic effect of 5-FU on the tumor cells (FIG. 17), whereas the three SOD mimetics [MnTBAP (FIG. 18), CuDIPS (FIG. 19) and MnDPDP (TESLASCAN, FIG. 20)] increase it.

2) Effects on Cell Viability:

The viability tests were carried out on the CT26, Hepa 16 and A549 tumor lines, and on normal human leukocytes, according to the protocol described in Example 2.

Oxaliplatin:

The oxaliplatin was used at a concentration of 10 μM in the case of the tumor cells, and at a concentration of 1 mM in the case of the normal leukocytes.

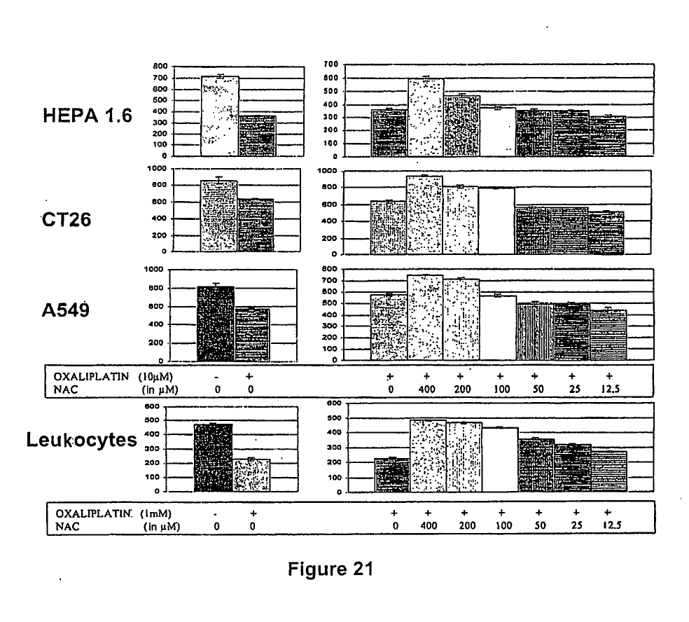

The results are illustrated by FIGS. 21, 22, 23 and 24, for NAC, MnTBAP, CuDIPS and MnDPDP, respectively.

Legend of FIGS. 21 to 24:

along the x-axis: presence (+) or absence (−) of oxaliplatin; concentration of antioxidant (in μM),

along the y-axis: OD at 550 nm—OD at 630 nm.

The treatment with oxaliplatin alone decreases, on average, the Hepa 1-6, CT26 and A549 tumor cell viability by 50%, 27% and 28%, respectively, and that of the normal leukocytes by approximately 50% (FIGS. 21 to 24).

NAC decreases, in a dose-dependent manner, the cytotoxic effects of oxaliplatin on all the tumor cell types, and on the normal leukocytes (FIG. 21).

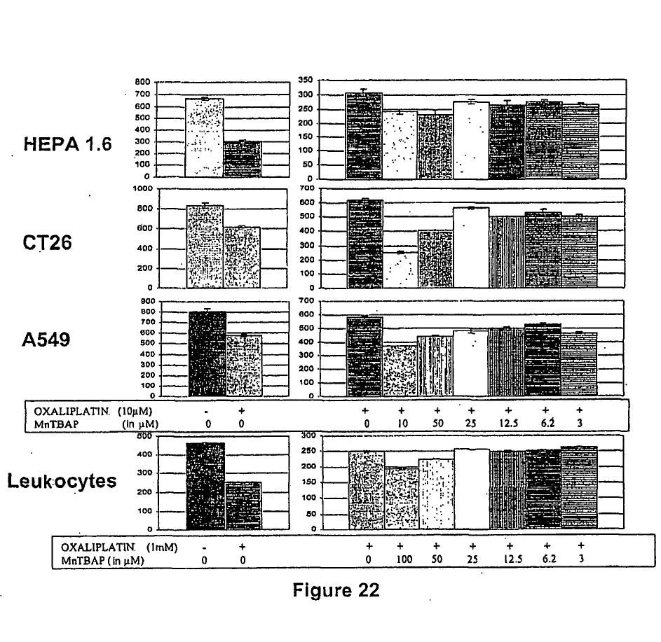

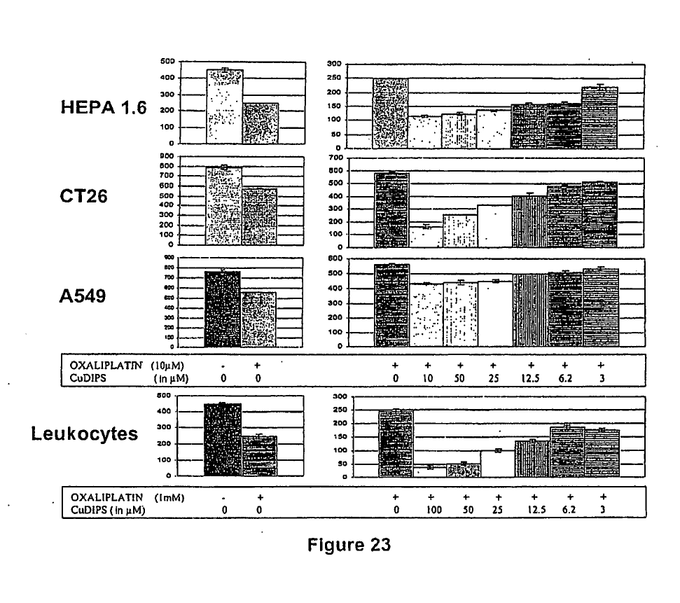

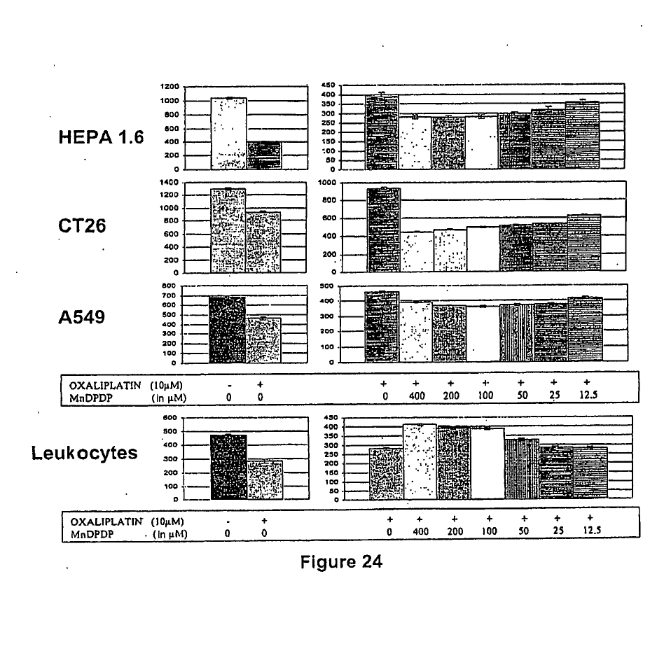

MnTBAP (FIG. 22), CuDIPS (FIG. 23) and MnDPDP (FIG. 24) increase, in a dose-dependent manner, the cytotoxic properties of oxaliplatin on the tumor cells.

On the normal leukocytes, MnTBAP (FIG. 22) and CuDIPS (FIG. 23) also increase the cytotoxic properties of oxaliplatin; on the other hand, MnDPDP (FIG. 24) inhibits, like NAC, the cytotoxic effect of oxaliplatin.

Taxol:

The taxol was used at a concentration of 10 μM in the case of the tumor cells, and at a concentration of 20 μM in the case of the normal leukocytes.

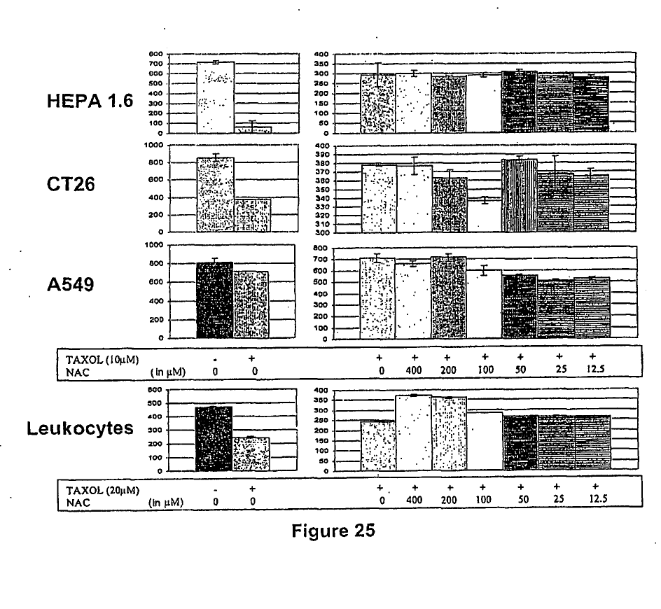

The results are illustrated by FIGS. 25, 26, 27 and 28, for NAC, MnTBAP, CuDIPS and MnDPDP, respectively.

Legend of FIGS. 25 to 28:

along the x-axis: presence (+) or absence (−) of taxol; concentration of antioxidant (in μM),

along the y-axis: OD at 550 nm—OD at 630 nm.

The treatment with taxol alone decreases, on average, the Hepa 1-6, CT26 and A549 tumor cell viability by 25%, 50% and 47%, respectively, and that of the normal leukocytes by approximately 50% (FIGS. 25 to 28).

The addition of NAC does not influence the cytotoxic activity of taxol on the tumor cells, and decreases it on the normal leukocytes (FIG. 25).

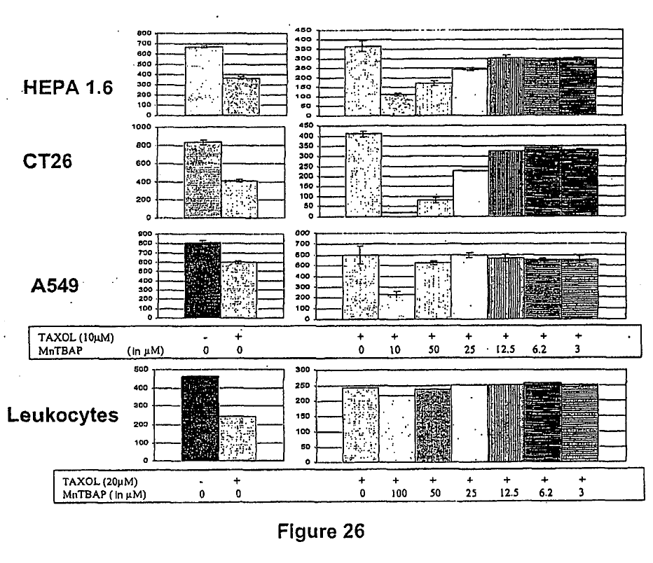

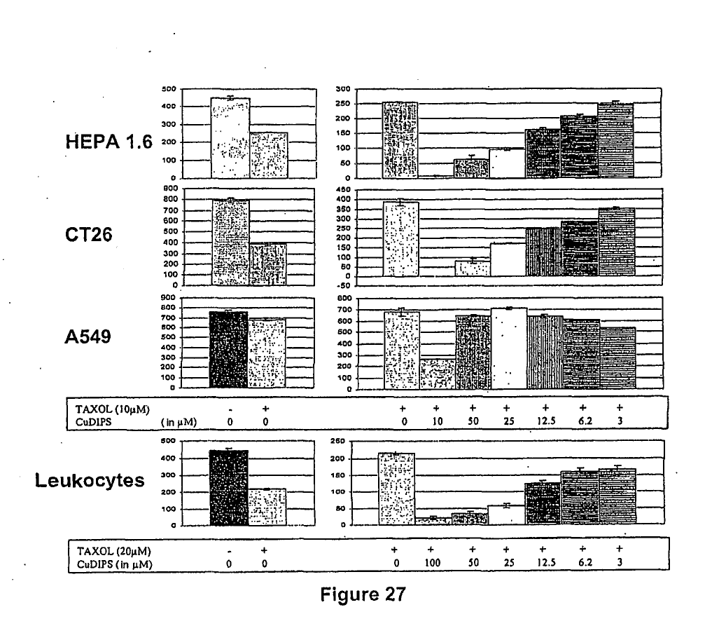

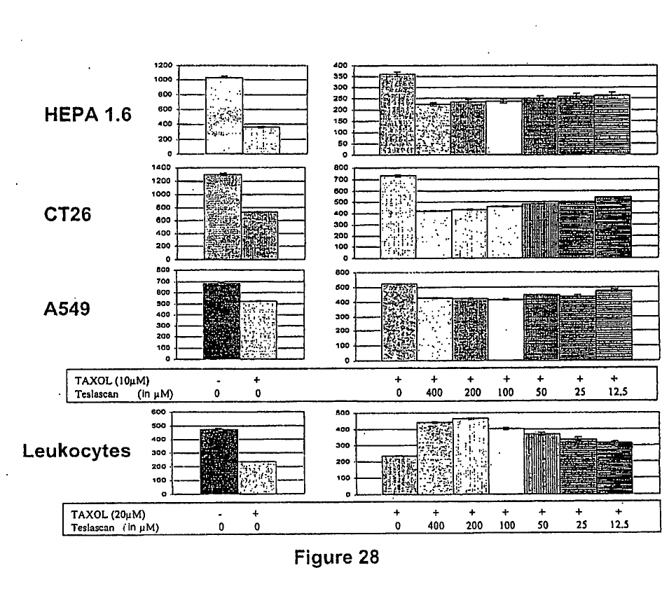

The addition of MnTBAP (FIG. 26), of CuDIPS (FIG. 27) or of MnDPDP (FIG. 28) increases the cytotoxic activity of taxol on the tumor cells. On the normal leukocytes, MnTBAP has virtually no influence on the cytotoxic effect of taxol (FIG. 26), and CuDIPS (FIG. 27) increases this cytotoxic effect; on the other hand, MnDPDP (FIG. 28) inhibits, like NAC, the cytotoxic effect of taxol.

5-Fluorouracil (5-FU):

The 5-FU was used at a concentration of 50 μM in the case of the tumor cells, and at a concentration of 40 mM in the case of the normal leukocytes.

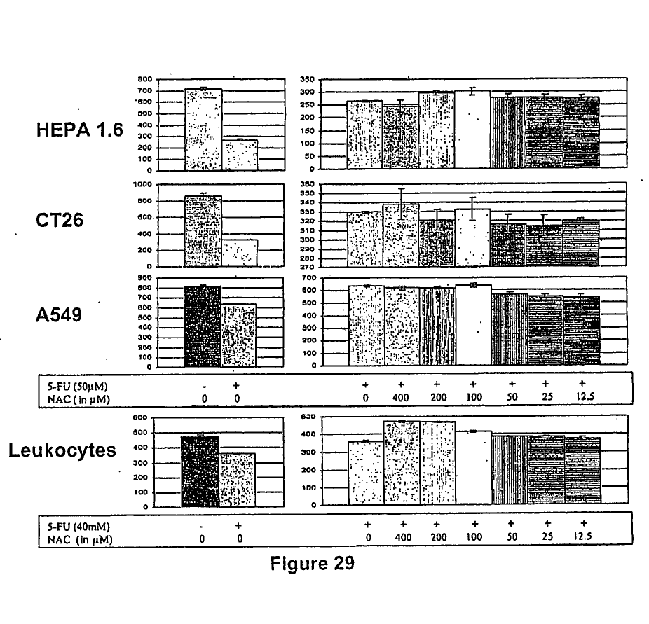

The results are illustrated by FIGS. 29, 30, 31 and 32, for NAC, MnTBAP, CuDIPS and MnDPDP, respectively.

Legend of FIGS. 29 to 32:

along the x-axis: presence (+) or absence (−) of 5-FU; concentration of antioxidant (in μM),

along the y-axis: OD at 550 nm—OD at 630 nm.

The treatment with 5-FU alone decreases, on average, the Hepa 1-6, CT26 and A549 tumor cell viability by 65%, 85% and 25%, respectively, and that of the normal leukocytes by approximately 19% (FIGS. 29 to 32).

The addition of NAC does not modify the cytotoxic activity of 5-FU on the tumor cells, and decreases it on the normal leukocytes (FIG. 29).

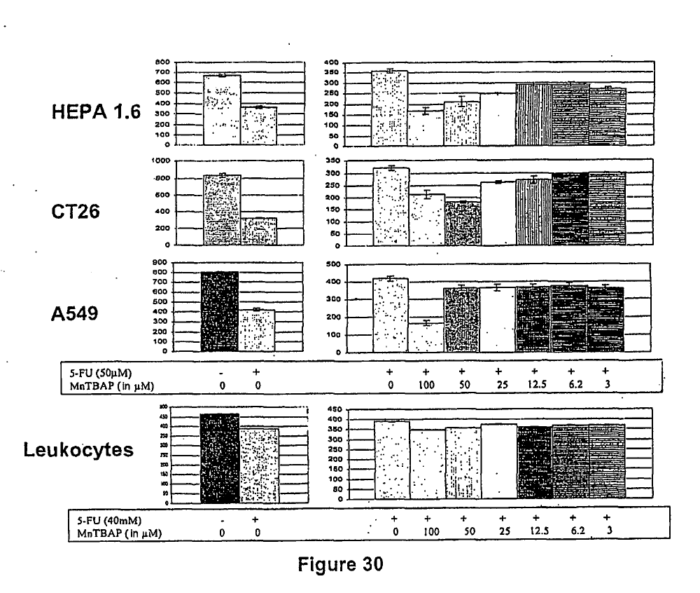

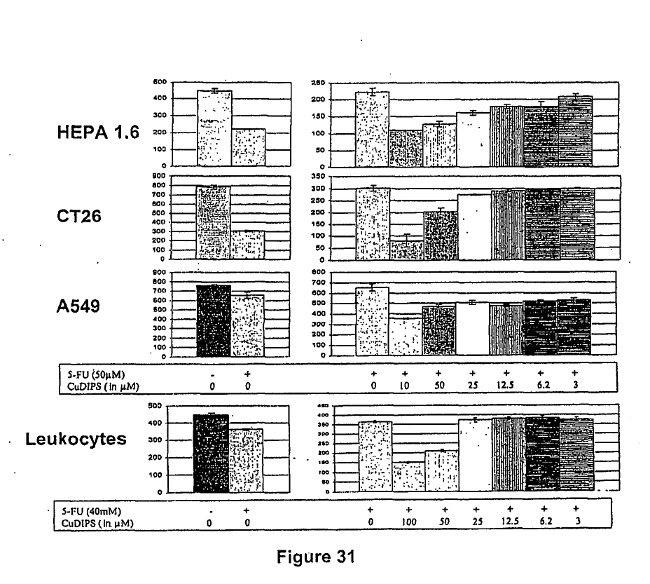

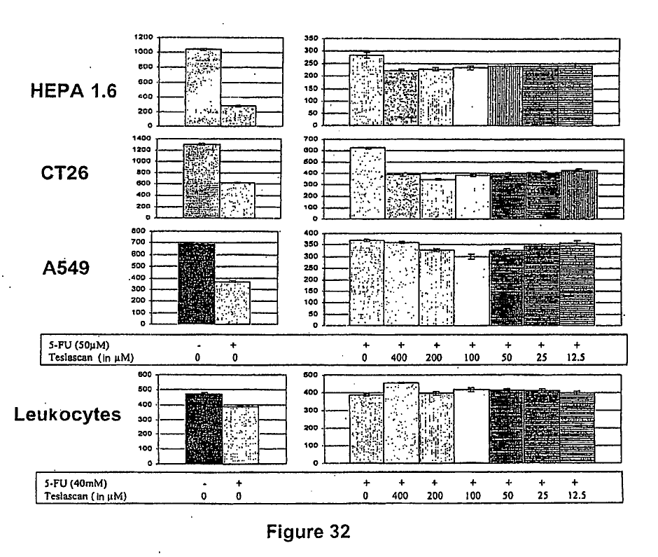

The addition of MnTBAP (FIG. 30), of CuDIPS (FIG. 31) and of MnDPDP (FIG. 32) increases the cytotoxic activity of 5-FU on the tumor cells. On the normal leukocytes, MnTBAP has only a very weak influence on the cytotoxic effect of 5-FU (FIG. 30); CuDIPS (FIG. 31) increases this cytotoxic effect; on the other hand, MnDPDP (FIG. 32) inhibits it.

### EXAMPLE 4

### Modulation of the Effects of Reactive Oxygen Species on DNA by NAC, CuDIPS, MnTBAP or MnDPDP

The DNA molecule is one of the main targets of the antitumor effect of platinum derivatives such as cisplatin or oxaliplatin. The platinum derivatives react on DNA by modifying its tertiary structure. Cationic metalloporphyrins are agents that are known to be able to interact with DNA.

It has recently been demonstrated that metalloporphyrins having SOD-mimicking properties can potentiate the harmful effects of ROSs on the structure of DNA.

The purified plasmid pcDNA3.1 (INVITROGEN) was used to analyze the potential DNA alterations in response to the addition of molecules used in cancer chemotherapy in the presence, or in the absence, of antioxidant enzyme modulators. This DNA was then stored at −20° C. in 10 mM TRIS, 1 mM EDTA until its use.

The plasmid DNA was incubated with oxaliplatin at a molar ratio of 0.50 in a final volume of 50 μl. MnTBAP (5 μM), CuDIPS (5 μM), mangafodipir (5 μM) or NAC (5 mM) were then added to the solution. The production of superoxide anion was realized by the addition of 200 μM xanthine (SIGMA) and 1 U of xanthine oxidase (SIGMA). The incubation was carried out in the dark at 37° C. for 24 h. At the end of the incubation period, 10 μl aliquots were subjected to 0.8% agarose gel electrophoresis and detected by ethidium bromide staining. The gels were then analyzed by densitometry (VILBER LOURMAT, Marnes-la-Vallée, France).

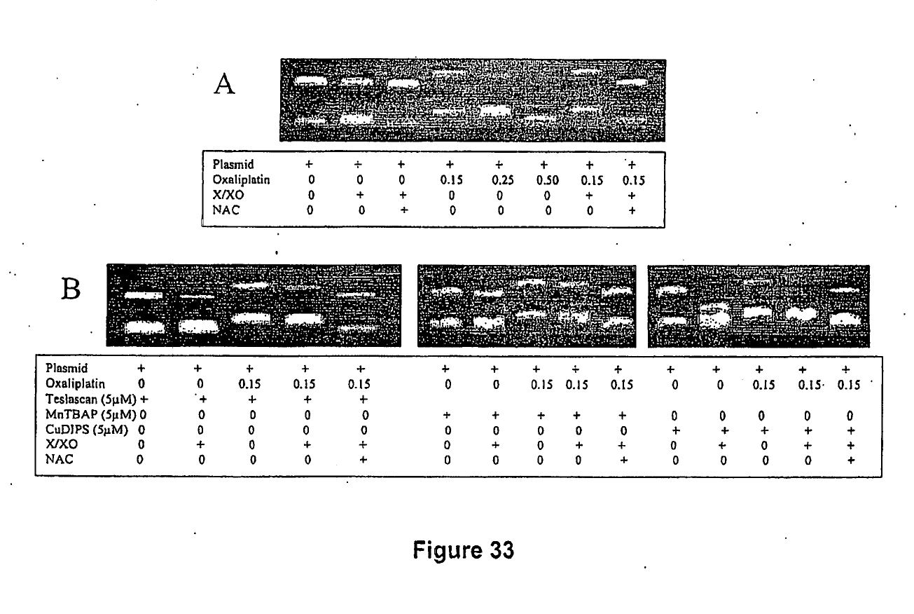

The results are given in FIG. 33.

Legend of FIG. 33:

A: presence (+) or absence (O) of plasmid; concentration of oxaliplatin (in μM); presence (+) or absence (0) of xanthine and of xanthine oxidase (X/XO); presence (+) or absence (0) of NAC.

B: presence (+) or absence (0) of plasmid; concentration of oxaliplatin (in μM); presence (+) or absence (0) of xanthine and of xanthine oxidase (X/XO); presence (+) or absence (0) of antioxidant (Teslascan, MnTBAP, CuDIPS or NAC).

The incubation of plasmid DNA with xanthine and xanthine oxidase (X/XO) generates superoxide anions which impair the native supercoiled form of DNA (form I DNA) and promote the circular form (form II DNA). The phenomenon is inhibited by ROS neutralization with NAC.

The incubation of plasmid DNA with oxaliplatin induces a dose-dependent impairment of the structure of the DNA which is at a maximum at the DNA/oxaliplatin ratio of 0.5. Under these conditions, the supercoiled form is no longer observed and a band corresponding to form III appears (linear form). The form I/form II ratio is reduced even further if the plasmid DNA is coincubated with the X/XO system and low doses of oxaliplatin. Incubation with NAC decreases the damage caused to the DNA.

In a second step, the effects of the SOD mimetics on the DNA impairments induced by oxaliplatin alone or oxaliplatin combined with ROSs were evaluated. Incubation of the plasmid DNA with mangafodipir induces, per se, DNA damage, as shown by the increase in the proportion of form II compared with the nontreated plasmid. This effect is amplified when either superoxide anions or oxaliplatin are added, and is at a maximum when mangafodipir, ROS and oxaliplatin are coincubated with the plasmid DNA. Here again, certain antioxidants such as NAC partially inhibit the DNA impairments.

A similar effect is observed when CuDIPS and, to a lesser extent, when MnTBAP, is used as SOD mimetic.

### EXAMPLE 7

### Antitumor Effects of NAC, of CuDIPS, of MnTBAP and of MnDPDP Combined or not Combined with Anticancer Chemotherapy in Mice

The in vivo antitumor activity of various antioxidant treatments was estimated. For these experiments, female six- to eight-week-old BALB/c (for the injection of CT-26 tumor cells) or C57/BL6 (for the injection of Hepa 1-6 tumor cells) mice were used (IFFA CREDO, L'Arbresles, France). Two million tumor cells were injected into the back of the animals subcutaneously. When the size of the tumor reached 200 to 500 mm³, the animals were given a single injection of 20 mg/kg of oxaliplatin (ELOXATIN®) or of a saline solution.

The mice were then treated intraperitoneally, two hours after the injection of oxaliplatin or of saline solution, with 10 mg/kg of mangafodipir, of MnTBAP or of CuDIPS, or with 150 mg/kg of NAC, or with a saline solution. The injection of the various antioxidants was continued for one month (three injections per week at the same doses). A group of mice inoculated with tumor cells was not treated.

The size of the tumors was measured every three days. The tumor volume was calculated as follows: VT (mm³)=(L×W²)/2, where L is the longest dimension and W the shortest dimension of the tumor in mm. Fifteen mice were included in each group.

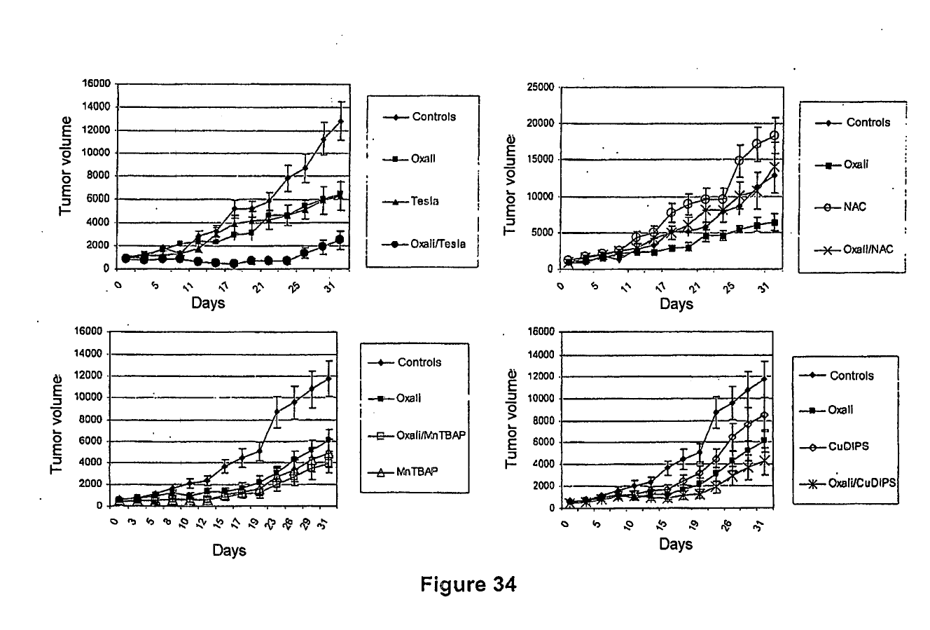

The results of the experiment based on the injection of CT26 carcinoma tumor cells into BALB/c mice are given in FIG. 34.

Legend to FIG. 34:
(♦) controls,
(▪) oxaliplatin,
(▴) teslascan,
(●) oxaliplatin+teslascan,
(◯) NAC,
(x) oxaliplatin+NAC,
(Δ) MnTBAP,
(□) oxaliplatin+MnTBAP,
(⋄) CuDIPS,
(*) oxaliplatin+CuDIPS.

The tumor volume is indicated along the y-axis; indicated along the x-axis is the number of days following the injection of oxaliplatin or of saline solution.

It is observed that injection of NAC into mice not treated with oxaliplatin induces a 44% increase in tumor volumes after one month, compared with the mice which do not receive NAC.

Whereas the administration of oxaliplatin divides the tumor volumes in half compared with the nontreated animals, the administration of NAC to mice treated with oxaliplatin completely blocks the inhibitory effect of oxaliplatin on tumor growth.

Conversely, the injection of chemical SOD mimetics such as MnTBAP, CuDIPS or mangafodipir decreases by 59%, 28% and 54%, respectively, the tumor volume at one month compared with the nontreated animals. In addition, the three SOD mimetics administered to mice treated with oxaliplatin decrease by 35%, 31% and 63%, respectively, the tumor volume at one month, compared with the animals treated only with oxaliplatin.

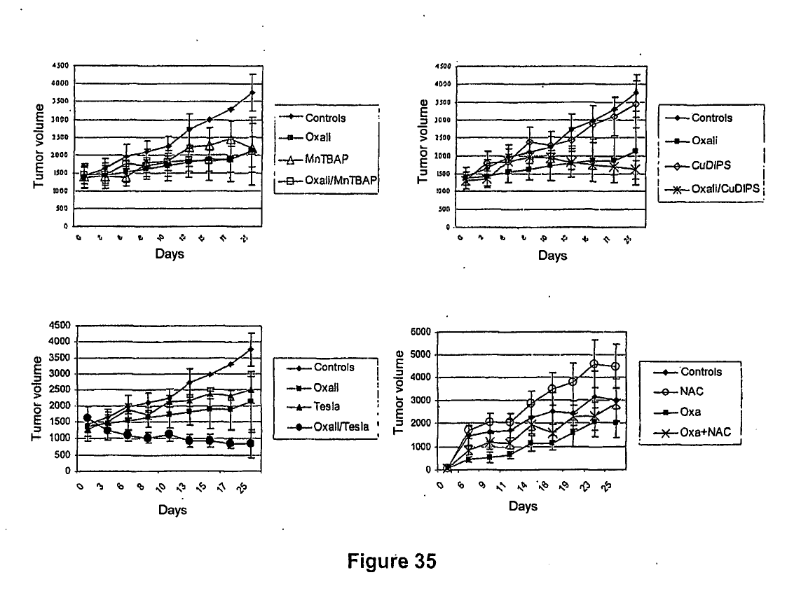

The results of the experiment based on the injection of Hepa 1-6 cells into C57BL/6 mice are given in FIG. 35.

Legend to FIG. 35:
(♦) controls,
(▪) oxaliplatin,
(▴) teslascan,
(●) oxaliplatin+teslascan,
(◯) NAC,
(x) oxaliplatin+NAC,
(Δ) MnTBAP,
(□) oxaliplatin+MnTBAP,
(⋄) CuDIPS,
(*) oxaliplatin+CuDIPS.

The tumor volume is indicated along the y-axis; indicated along the x-axis is the number of days following the injection of oxaliplatin or of saline solution.

Here again, it is observed that the injection of NAC induces a 50% increase in tumor volumes after one month, compared with the mice which are not given NAC. Whereas the administration of oxaliplatin divides the tumor volumes by four compared with the nontreated animals, the administration of NAC to mice treated with oxaliplatin completely blocks the inhibitory effect of oxaliplatin on tumor growth. Conversely, the injection of chemical SOD mimetics such as MnTBAP, CuDIPS or mangafodipir decreases by 42%, 9% and 34%, respectively, the tumor volume at one month compared with the nontreated animals. In addition, while the coadministration of MnTBAP and of CuDIPS with oxaliplatin does not significantly increase the antitumor effect of oxaliplatin, the administration of MnDPDP to mice treated with oxaliplatin decreases by 63% the tumor volume at one month, compared with the animals treated only with oxaliplatin (FIG. 35).

1. An anticancer medicinal product comprising an antitumor and leukocyte-protecting active ingredient and an antitumor-and-leukocyte-protecting amount of a superoxide dismutase and glutathione reductase mimetic.

2. The product claimed in claim 1, wherein said mimetic is mangafodipir.

3. The product as claimed in claim 1, wherein said mimetic is used in combination with an antitumor agent capable of inducing a reactive oxygen species production in cells.

4. The product as claimed in claim 3, wherein said antitumor agent is chosen from doxorubicin, mitomycin C, etoposide, platinum derivatives, tamoxifen, taxol, 5-fluorouracil, irinotecan, gemcitabine, endoxan, streptozotocin, bleomycin and vincristine.

5. A pharmaceutical composition comprising a superoxide dismutase and glutathione reductase mimemtic, combined with an antitumor agent chosen from oxaliplatin, 5-fluorouracil and taxol.

6. The pharmaceutical composition as claimed in claim 5, wherein said mimetic is mangafodipir.
